(19) United States
(12) Patent Application Publication
(10) Pub. No.: US 2016/0089130 A1
(43) Pub. Date: Mar. 31, 2016

(54) Soft Tissue Attachment

(71) Applicant: Biomet Sports Medicine, LLC, Warsaw, IN (US)

(72) Inventors: Jacy C. HOEPPNER, Warsaw, IN (US); Adam FINLEY, Winona Lake, IN (US); Kevin T. STONE, Winona Lake, IN (US)

(21) Appl. No.: 14/502,171

(22) Filed: Sep. 30, 2014

(51) Int. Cl. A61B 17/04

(52) U.S. Cl. CPC A61B 17/0401; A61B 2017/0427; A61B 2017/042; A61B 2017/0445; A61B 2017/0464

(57) ABSTRACT

A suture attachment device and method for securing a suture to an anatomic structure. The suture attachment device includes a cannulated member including a first surface and a second surface. A plurality of castellations extend axially outward from at least one of the first surface and the second surface, wherein each castellation includes a proximal end connected to the cannulated member, and a distal end located distal from the cannulated member, the distal end being flared radially outward relative to the proximal end.

### FIELD

The present disclosure relates to soft tissue attachment device.

### BACKGROUND

This section provides background information related to the present disclosure which is not necessarily prior art.

When soft tissue tears away or separates from bone, reattachment becomes necessary. Various devices, including sutures alone, screws, staples, wedges, and plugs have been used to secure soft tissue to bone. Anchors have been developed that are designed to be inserted into a pre-drilled hole. Other anchors are self-tapping. All of the above-noted anchors rely on a flight of threads disposed on the outer surface of a shank to secure it to the bone. They all provide a relatively easy method of fixation in hard, cortical bone.

While the above-noted anchors work well for their intended purpose, improvements to the anchors may be beneficial in various areas. For example, improvements can be directed to an anchor's “pull-out” strength, and Improvements can be made to further reduce any tendency of the anchor to “back out” of the implantation site. Yet another improvement can be made to reduce any bone loss that may be incurred upon installation of the anchors.

Accordingly, a need exists for an anchor that can be secured easily and effectively. A need also exists for an anchor that displaces a minimum amount of bone upon insertion. In addition, a need exists for an anchor having exceptional pull-out strength, and also averse to “backing out” of the ins ion site.

### SUMMARY

This section provides a general summary of the disclosure, and is not a comprehensive disclosure of its full scope or all of its features.

The present disclosure provides a suture attachment device for securing a suture to an anatomic structure. The suture attachment device includes a cannulated member including a first surface and a second surface; and a plurality of castellations extending axially outward from at least one of the first surface and the second surface, wherein each castellation includes a proximal end connected to the cannulated member, and a distal end located distal from the cannulated member, the distal end being flared radially outward relative to the proximal end.

The present disclosure also provides a method of securing at least one suture to an anatomic structure. The method includes forming an aperture in the anatomic structure, and passing a suture through a cannulated member of a suture attachment device. The cannulated member includes a first surface and a second surface, and a plurality of prongs extending axially outward from at least one of the first surface and the second surface. Each prong includes a proximal end connected to the cannulated member, and a distal end located distal from the cannulated member. The distal end is flared radially outward relative to the proximal end. The method also includes knotting the suture to secure the suture to the suture attachment device, and passing the suture attachment device with the suture secured thereto through the aperture, wherein as the suture attachment device passes through the aperture, the prongs radially deflect inward, and after passing through the aperture, the prongs radially deflect outward and engage the anatomic structure to secure the suture to the anatomic structure.

The present disclosure also provides a suture attachment device for securing a suture to bone, including a cannulated member including a first surface and a second surface; a plurality of deflectable first prongs extending axially and flaring radially outward from the first surface, the plurality of first prongs defining a sharpened end of the suture attachment device; and a plurality of second prongs extending axially outward from the second surface, the plurality of second prongs defining a blunt end of the suture attachment device. The plurality of first prongs and the plurality of second prongs define nesting features for engaging a plurality of the suture attachment devices together.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a suture attachment device according to a principle of the present disclosure;

FIG. 2 is a projected view of the suture attachment device of FIG. 1, in an unwrapped state;

FIG. 3 is a top-perspective view of the suture attachment device of FIG. 1;

FIG. 4 is a bottom-perspective view of the suture attachment device of FIG. 1;

FIG. 5 is a side-perspective view of the suture attachment device of FIG. 1;

FIG. 6 is a perspective view of another suture attachment device according to a principle of the present disclosure;

FIG. 7 is a projected view of the suture attachment device of FIG. 6, in an unwrapped state;

FIG. 8 is a top-perspective view of the suture attachment device of FIG. 6;

FIG. 9 is a bottom-perspective view of the suture attachment device of FIG. 6;

FIG. 10 is a side-perspective view of the suture attachment device of FIG. 6;

FIG. 11 illustrates an exemplary suture attachment device according to a principle of the present disclosure securing a suture to a hard tissue anatomic structure such as bone;

FIG. 12 illustrates an exemplary suture attachment device according to a principle of the present disclosure securing a suture to a soft tissue anatomic structure such as muscle;

FIG. 13 illustrates a plurality of exemplary suture attachment devices according to a principle of the present disclosure securing a suture to an anatomic structure;

FIG. 14 illustrates the plurality of exemplary suture attachment devices in FIG. 13 nested together;

FIG. 15 illustrates a plurality of exemplary suture attachment devices according to a principle of the present disclosure securing a plurality of sutures to an anatomic structure; and

FIG. 16 illustrates another suture attachment device according to a principle of the present disclosure

Corresponding reference numerals indicate corresponding parts throughout the several views of the drawings.

### DETAILED DESCRIPTION

Example embodiments will now be described more fully with reference to the accompanying drawings.

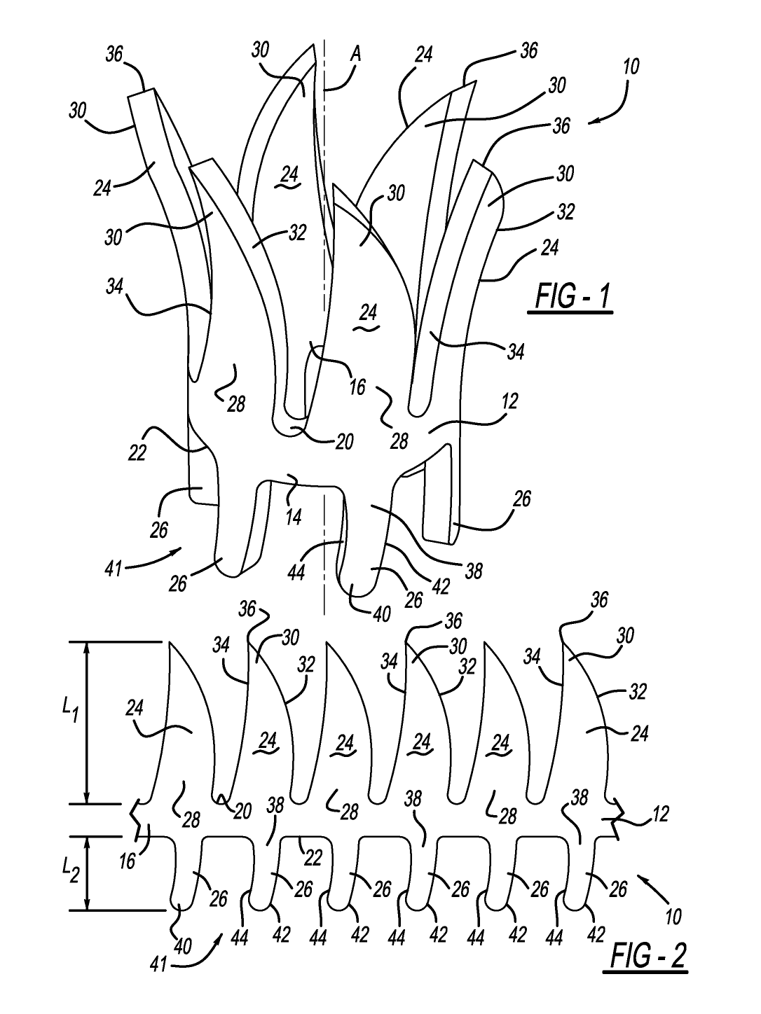

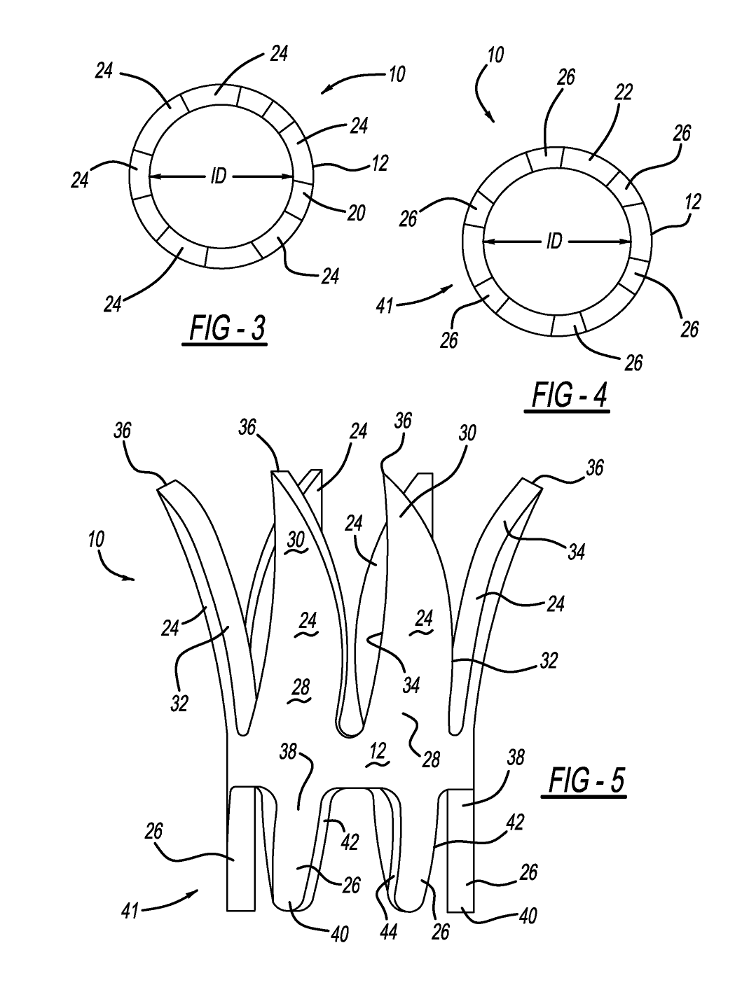

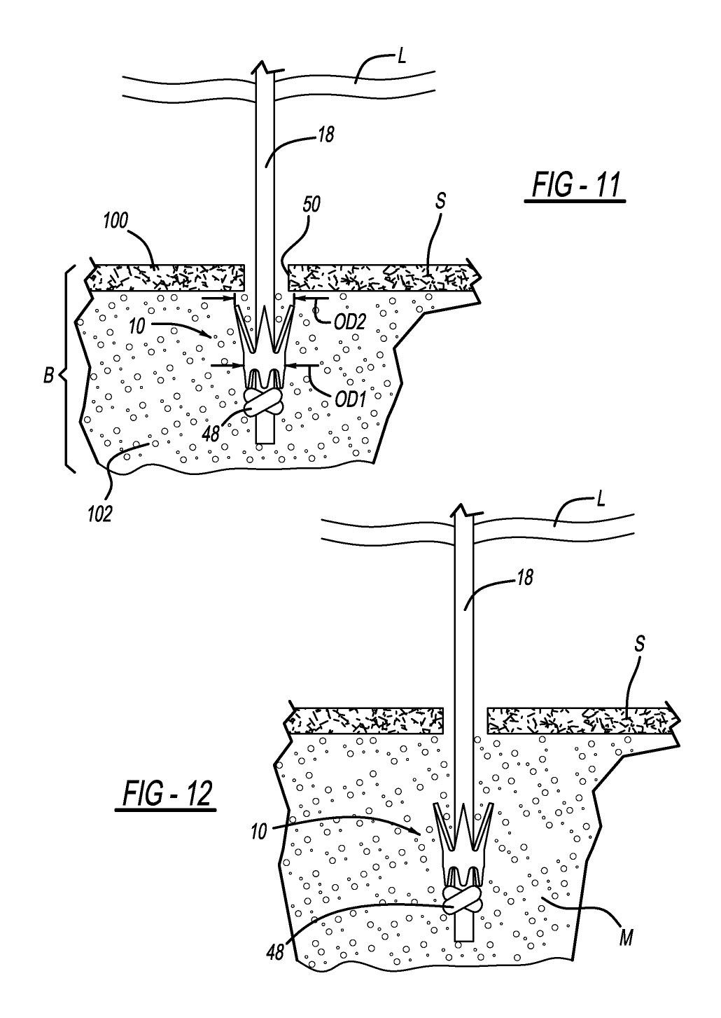

FIGS. 1-5 illustrate a suture attachment device 10 according to a first exemplary embodiment according to the present disclosure. Although the present disclosure references the attachment of a suture to an anatomic structure, it should be understood that the attachment device 10 may be used to attach a wire or some other anchoring means without departing from the scope of the present disclosure. Suture attachment device 10 includes a cylindrical annular ring 12. Cylindrical ring 12 includes an exterior surface 14 and an interior surface 16 such that suture attachment device 10 is cannulated and allows suture 18 (FIG. 11) to pass therethrough along axis A. Cylindrical ring 12 includes a first or upper surface 20 and a second or lower surface 22. Suture attachment device 10 is crown-shaped such that a plurality of first castellations or first prongs 24 extend axially outward from first surface 20. First prongs 24 also flare radially outward from axis A such that first prongs 24 are curved. First prongs 24 are also resilient so as to be deflectable. Although not required by the present disclosure, a plurality of second castellations or second prongs 26 may extend axially outward from second surface 22. First prongs 24 are configured to engage an anatomic structure S such as bone, ligament, tendon, muscle, or some other tissue after the suture 18 has been passed through the anatomic structure S. In addition, each of first prongs 24 and second prongs 26 are designed to allow for nesting between multiple suture attachment devices 10, as will be described in more detail below.

First prongs 24 each include a proximal base end 28 connected to or unitary with first surface 20, and a distal pointed end 30 distal from first surface 20. Distal pointed ends 30 may be sharp to engage and fix suture attachment device 10 to anatomic structure S. Each prong 24 includes a first tapered surface 32 and a second tapered surface 34 that extend from proximal end 28 to distal end 30 and terminate at an apex 36 such that first prongs 24 have a length L1. As noted above, apex 36 can be sharp to engage and fix suture attachment device 10 to anatomic structure S. In the illustrated embodiment, first and second tapered surfaces 32 and 34 each include a radius of curvature such that first prongs 24 are fin-shaped. In addition, in the illustrated embodiment, first tapered surface 32 includes a greater radius of curvature in comparison to second tapered surface 34. It should be appreciated, however, that first and second tapered surfaces 32 and 34 can include the same radius of curvature, or that second tapered surface 34 can include a greater radius of curvature in comparison to first tapered surface 32 without departing from the scope of the present disclosure. Furthermore, it should be appreciated that first prongs 24 flare radially outward from cylindrical ring 12 such that an outer diameter OD2 of suture attachment device 10 defined by distal ends 30 is greater than the outer diameter OD1 of cylindrical ring 12. Although six first prongs 24 are illustrated in FIG. 1 at sixty degree intervals around cylindrical ring 12, it should be understood that any number of first prongs 24 is contemplated. For example, configurations including sets of two, three, four, and five first prongs 24 are contemplated.

Second prongs 26 are similar to first prongs 24 in that each second prong 26 includes a proximal base end 38 connected to or unitary with second surface 22, and a distal end 40 distal from second surface 20. Each prong 26 includes a first tapered surface 42 and a second tapered surface 44 that extend from proximal end 38 to distal end 40 and terminate at a terminal end 46 such that second prongs 26 have a length L2. In the illustrated embodiment, L1 is greater than L2. It should be understood, however, that L1 can be equal to L2, or than L2 may be greater than L1 without departing from the scope of the present disclosure. First and second tapered surfaces 42 and 44 each include a radius of curvature such that second prongs 26 are fin-shaped. In addition, in the illustrated embodiment, first and second tapered surfaces 42 and 44 have an equal radius of curvature such that second prongs 26 are slightly curved. It should be appreciated, however, that first and second tapered surfaces 32 and 34 can include the different radii of curvature without departing from the scope of the present disclosure. Further, in contrast to first prongs 24, second prongs 26 do not flare radially outward relative to cylindrical ring 12 and are not sharpened at distal ends 40. Rather, second prongs 26 simply extend axially relative to cylindrical ring 12 and define a blunt end 41 of suture attachment device 10 to assist with insertion of second prongs 26 into anatomic structure S, as will be described in more detail below. Further, as noted above, the use of second prongs 26 allows for nesting between multiple suture attachment devices 10 at a single attachment site of anatomic structure S. In this regard, the spaces between adjacent first prongs 24 and adjacent second prongs 26 can be sized to allow for first prongs 24 or second prongs 26 of another suture attachment device 10 to engage therewith. Although six second prongs 26 are illustrated in FIG. 1 at sixty degree intervals around cylindrical ring 12, it should be understood that any number of second prongs 24 is contemplated. For example, configurations including zero, two, three, four, and five second prongs 26 are contemplated.

To secure suture 18 to the anatomic structure S, suture 18 is first passed through cylindrical ring 12 in the direction from first prongs 24 to second prongs 26. After passing through cylindrical ring 12, suture 18 is tied to form a knot 48 to prevent suture 18 from passing back through cylindrical ring 12. In this regard, an inner diameter ID of cylindrical ring 12 is slightly larger than the diameter of suture 18. For example, if suture 18 has a diameter of 1 mm, inner diameter ID of cylindrical ring 12 can be approximately 1.25 mm to allow suture 18 to freely pass through cylindrical ring 12 before knotting and prevent suture 18 from passing back through cylindrical ring 12 after being knotted.

After suture 18 is secured to relative to suture attachment device 10, suture attachment device 10 including suture 18 is inserted into a pre-drilled aperture or bore 50 formed in the anatomic structure S. In the illustrated embodiment, a ligament L is being attached to an anatomic structure S that is a bone B including a cortical bone layer 100 and a cancellous bone layer 102, with pre-drilled aperture 50 being formed in cortical bone layer 100. The pre-drilled aperture 50 has a diameter substantially equal to an outer diameter OD of cylindrical ring 12. Specifically, second prongs 26 are aligned with aperture 50 and suture attachment device 10 is pressed through aperture 50 using an insertion device (not shown). As suture attachment device 10 is being pressed through aperture 50, first prongs 24 will begin to deflect radially inward. That is, as noted above, first prongs 24 are flared radially outward relative to cylindrical ring 12, and first prongs 24 are also deflectable. Thus, as suture attachment device 10 is being inserted through aperture 50, first prongs 24 can deflect radially inward to allow suture attachment device 10 to pass entirely through aperture 50. After passing entirely through aperture 50, first prongs 24 will deflect radially outward to their original flared position within cancellous bone layer 102. Because cancellous bone layer 102 is porous and “spongy” relative to cortical bone layer 100, first prongs 24 are able to deflect outward and engage with either cortical bone layer 100 or pores within cancellous bone layer 102 to lock suture attachment device 10 within the bone B. That is, because first prongs 24 will return to their original flared position, apexes 36 are free to engage the bone B as suture 18 is tightened such that suture attachment device 10 acts like an anchor. In this manner, suture 18 is securely fastened to anatomic structure S to secure ligament L to bone B.

It should be understood that when suture attachment device 10 is being used to secure suture 18 to a soft tissue such as muscle, the formation of aperture 50 is not necessary. More specifically, referring to FIG. 12, it can be seen that anatomic structure S is a muscle M, and that suture attachment device 10 including suture 18 has merely been pressed into muscle M such that no pre-drilled aperture 50 is necessary. In this instance, first prongs 24 may still deflect radially inward to allow suture attachment device 10 to pass entirely into muscle M. After entering the soft tissue such as muscle M, first prongs 24 will deflect radially outward to their original flared position within the soft tissue such as muscle M.

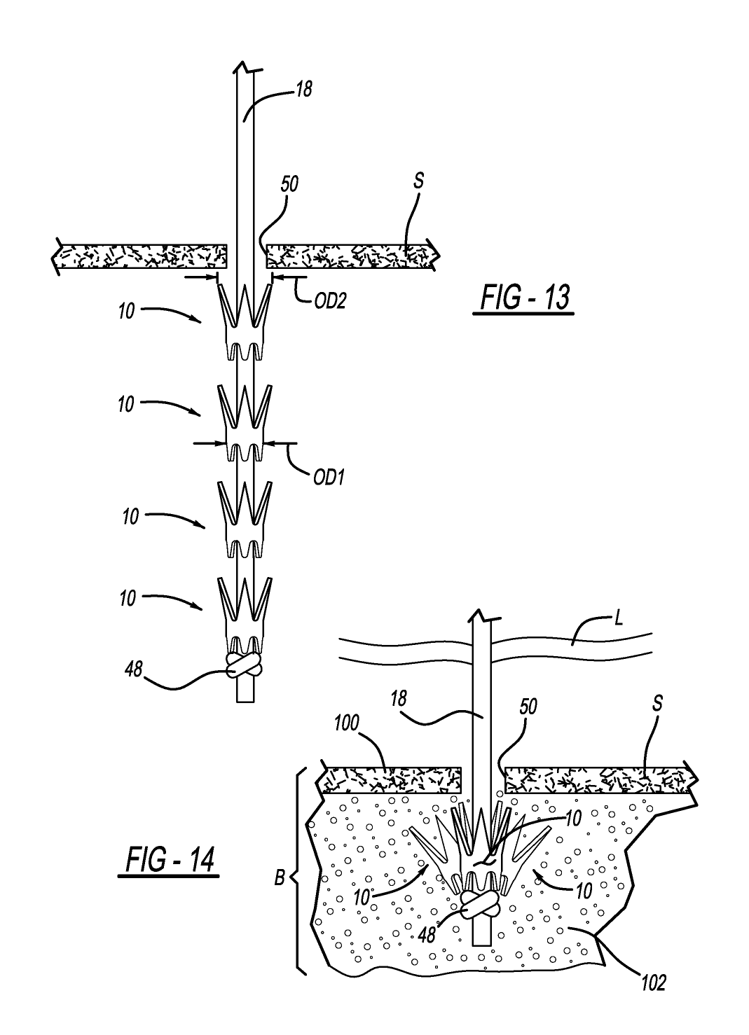

As illustrated in FIG. 13, it should be understood that suture 18 can be passed through a plurality of suture attachment devices 10 before being knotted. After passing suture 18 and each suture attachment device 10 through aperture 50, and while suture 18 is being tightened relative to anatomic structure S, the suture attachment devices 10 may begin to bunch up (FIG. 13). As the suture attachment devices 10 begin to bunch up, first and second prongs 24 and 26 begin to act as nesting features that allow attachment devices 10 to nest with each other. That is, first and second prongs 24 and 26 may mate with first and second prongs 24 and 26 of adjacent suture attachment devices 10 to form a larger “anchor” that secures suture 18 to anatomic structure S. In this manner, a more robust attachment of suture 18 to anatomic structure S can be made.

Although each suture attachment device 10 illustrated in FIG. 13 is the same size, it should be understood that differently sized suture attachment devices 10 could be used. For example, a larger suture attachment device 10 could be located proximate anatomic structure S, and progressively smaller suture attachment devices 10 could be used as the distance from anatomic structure S increases. Alternatively, a suture attachment device 10 could be used proximate anatomic structure S and progressively larger suture attachment devices 10 could be used as the distance from anatomic structure S increases.

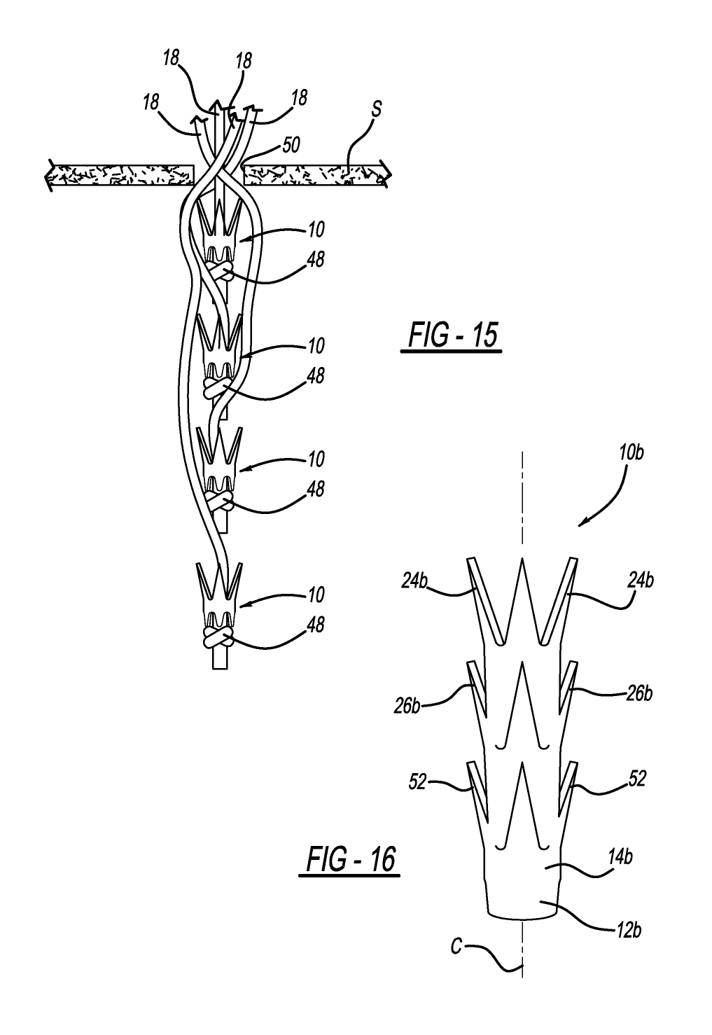

It should also be understood that, as illustrated in FIG. 15, a plurality of sutures 18 each having a suture attachment device 10 can be passed through aperture 50. After passing each suture 18 including a respective suture attachment device 10 through aperture 50, and while each suture 18 is being tightened relative to anatomic structure S, the suture attachment devices 10 may begin to bunch up (see, e.g., FIG. 14). As the suture attachment devices 10 begin to bunch up, first and second prongs 24 and 26 begin to act as nesting features that allow attachment devices 10 to nest with each other. That is, first and second prongs 24 and 26 may mate with first and second prongs 24 and 26 of adjacent suture attachment devices 10 to form a larger “anchor” that secures suture 18 to anatomic structure S. In this manner, a more robust attachment of sutures 18 to anatomic structure S can be made.

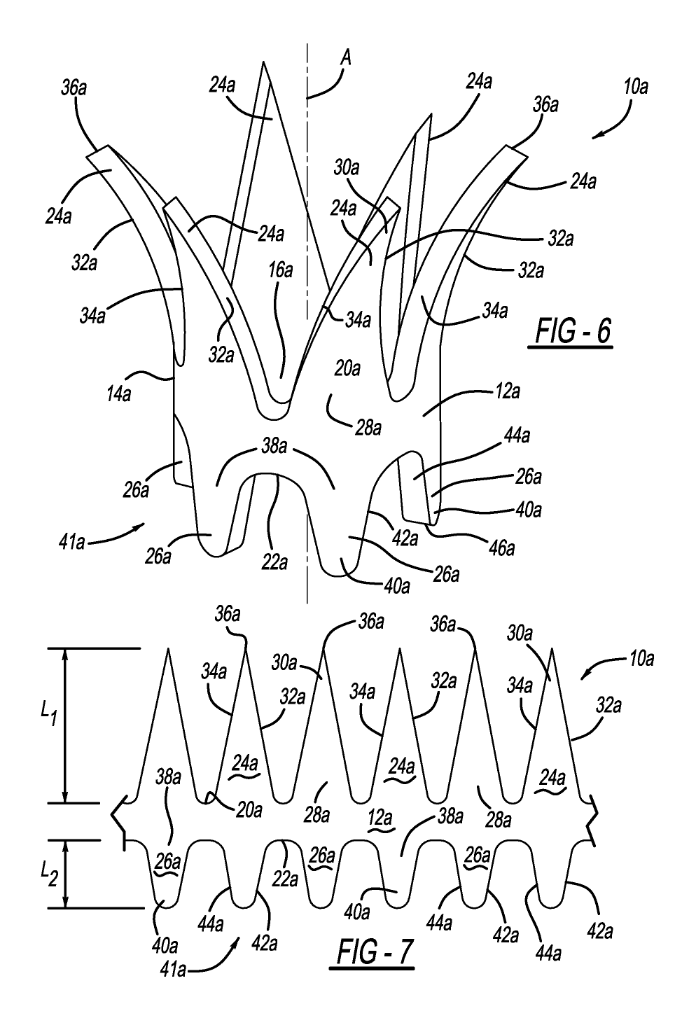

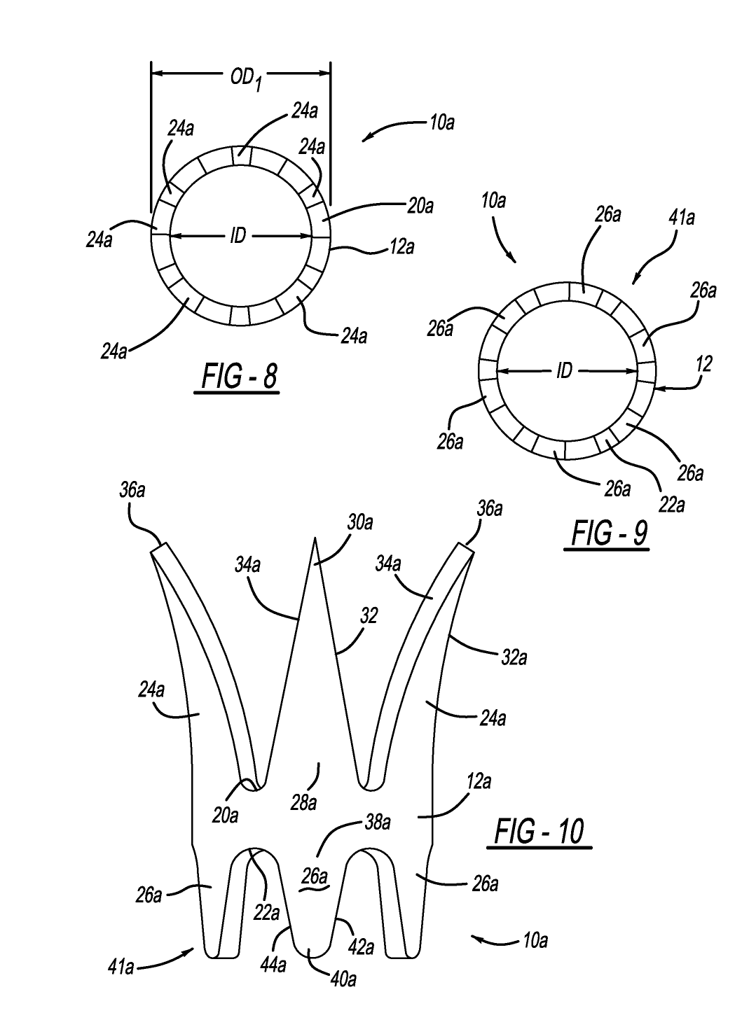

Now referring to FIGS. 6 to 10, a second exemplary embodiment will be described. Suture attachment device 10a includes a cylindrical annular ring 12a. Cylindrical ring 12a includes an exterior surface 14a and an interior surface 16a such that suture attachment device 10a is cannulated and allows suture 18 (FIG. 11) to pass therethrough along axis B. Cylindrical ring 12a includes a first or upper surface 20a and a second or lower surface 22a. A plurality of first castellations or first prongs 24a extend axially outward from first surface 20a such that suture attachment device 10a is crown-shaped. First prongs 24a also flare radially outward from axis B such that first prongs 24a are curved. First prongs 24a are also resilient so as to be deflectable. Although not required by the present disclosure, a plurality of second castellations or second prongs 26a may extend axially outward from second surface 22a. First prongs 24a are configured to engage an anatomic structure S such as bone or ligament after the suture 18 has been passed through the anatomic structure.

First prongs 24a each include a proximal base end 28a connected to or unitary with first surface 20a, and a distal end 30a distal from first surface 20a. Each prong 24a includes a first tapered surface 32a and a second tapered surface 34a that extend from proximal end 28a to distal end 30a and terminate at an apex 36a such that first prongs 24a have a length L1. Apex 36a can be sharp to engage and fix suture attachment device 10 to anatomic structure S. In the illustrated embodiment, first and second tapered surfaces 32a and 34a are linear such that first prongs 24a are triangle-shaped.

First prongs 24a flare radially outward from cylindrical ring 12a such that an outer diameter OD2 of suture attachment device 10a defined by distal ends 30a is greater than the outer diameter OD1 of cylindrical ring 12a. Although six first prongs 24a are illustrated in FIG. 6 at sixty degree intervals around cylindrical ring 12a, it should be understood that any number of first prongs 24a is contemplated. For example, configurations including sets of two, three, four, and five first prongs 24a are contemplated.

Second prongs 26a are similar to first prongs 24a in that each second prong 26a includes a proximal end 38a connected to or unitary with second surface 22a, and a distal end 40a distal from second surface 20a. Each prong 26a includes a first tapered surface 42a and a second tapered surface 44a that extend from proximal end 38a to distal end 40a and terminate at a terminal end 46a such that second prongs 26a have a length L2. In the illustrated embodiment, L1 is greater than L2. It should be understood, however, that L1 can be equal to L2, or than L2 may be greater than L1 without departing from the scope of the present disclosure. In the illustrated embodiment, first and second tapered surfaces 42a and 44a are linear such that second prongs 26a are essentially triangle-shaped. It should be noted, however, that terminal end 46a is rounded rather than defining a sharp point, which assists in inserting suture attachment device 10a through aperture 50. Further, in contrast to first prongs 24a, second prongs 26a do not flare radially outward relative to cylindrical ring 12a. Rather, second prongs 26a simply extend axially relative to cylindrical ring 12a and define a blunt end 41a of suture attachment device 10 to assist with insertion of second prongs 26a into anatomic structure S, as was discussed in detail above. Further, as noted above, the use of second prongs 26a allows for nesting between multiple suture attachment devices 10a at a single attachment site of anatomic structure S. In this regard, the spaces between adjacent first prongs 24a and adjacent second prongs 26a can be sized to allow for first prongs 24a or second prongs 26a of another suture attachment device 10a to engage therewith. Although six second prongs 26a are illustrated in FIG. 6 at sixty degree intervals around cylindrical ring 12a, it should be understood that any number of second prongs 26a is contemplated. For example, configurations including zero, two, three, four, and five second prongs 26a are contemplated.

Now referring to FIG. 16, a suture attachment device 10b is illustrated. Suture attachment device 10b includes a cylindrical annular ring 12b. Cylindrical ring 12b includes an exterior surface 14b and an interior surface (not shown) such that suture attachment device 10b is cannulated and allows suture 18 (FIG. 11) to pass therethrough along axis C. Suture attachment device 10b includes a plurality of first castellations or first prongs 24b such that suture attachment device 10b is crown-shaped. First prongs 24b also flare radially outward from axis C such that first prongs 24b are curved. First prongs 24b are also resilient so as to be deflectable.

Suture attachment device 10b also includes a plurality of second castellations or second prongs 26b. Second prongs 26b are similar to first prongs 24b in that each second prong 26b also flares radially outward from axis C such that second prongs 26b are curved. Second prongs 24b are also resilient so as to be deflectable.

Suture attachment device 10b also includes a plurality of third castellations or third prongs 52. Third prongs 52 are similar to first and second prongs 24b and 26b in that each third prong 52 also flares radially outward from axis C such that third prongs 52 are curved. Third prongs 52 are also resilient so as to be deflectable. The use of first 24b, second 26b, and third prongs 52 increase the pull-out strength of suture attachment device 10b. Although annular ring 12b has been described as cylindrical, annular ring 12b may be cone-shaped or tapered without departing from the scope of the present disclosure.

In each of the exemplary embodiments, suture attachment devices 10 and 10a may be formed from materials such as stainless steel, titanium or titanium alloy, nitanol, resorbable magnesium, polyether ether ketone (PEEK), or polyethylene. To form suture attachment devices 10 and 10a, a cylindrical tube may be laser processed to form the first and second prongs. Alternatively, suture attachment devices 10 and 10 may be micro-molded, or formed using 3D printing techniques.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A suture attachment device for securing a suture to an anatomic structure, comprising:
a cannulated member including a first surface and a second surface; and
a plurality of castellations extending axially outward from at least one of the first surface and the second surface,
wherein each castellation includes a proximal end connected to the cannulated member, and a distal end located distal from the cannulated member.

2. The suture attachment device according to claim 1, wherein each castellation includes a first tapered surface and a second tapered surface that extend from the proximal end to the distal end and terminate at an apex.

3. The suture attachment device according to claim 2, wherein the first tapered surface and the second tapered surface each include a radius of curvature.

4. The suture attachment device according to claim 3, wherein the second tapered surface includes a greater radius of curvature than the first tapered surface.

5. The suture attachment device according to claim 1, wherein the plurality of castellations extends from each of the first and second surfaces.

6. The suture attachment device according to claim 5, wherein a plurality of first castellations extend from the first surface, and a plurality of second castellations extend from the second surface, the distal ends of the first castellations are flared radially outward relative to the proximal ends, and the second castellations are not flared radially outward relative to the proximal ends thereof.

7. The suture attachment device according to claim 6, wherein each castellation of the first and second castellations include a first tapered surface and a second tapered surface that extend from the proximal end to the distal end and terminate at an apex.

8. The suture attachment device according to claim 7, wherein the first tapered surface and the second tapered surface each include a radius of curvature.

9. The suture attachment device according to claim 8, wherein the second tapered surface includes a greater radius of curvature than the first tapered surface.

10. The suture attachment device according to claim 6, wherein the first castellations have a greater length than the second castellations.

11. The suture attachment device according to claim 6, wherein each of the first and second castellations are fin-shaped.

12. The suture attachment device according to claim 7, wherein each of the first and second tapered surfaces of the first castellations are linear.

13. A method of securing at least one suture to an anatomic structure, comprising:
passing a suture through a cannulated member of a suture attachment device, the cannulated member including a first surface and a second surface, and a plurality of prongs extending axially outward from at least one of the first surface and the second surface, wherein each prong includes a proximal end connected to the cannulated member, and a distal end located distal from the cannulated member, the distal end being flared radially outward relative to the proximal end;
knotting the suture to secure the suture to the suture attachment device; and
passing the suture attachment device with the suture secured thereto into the anatomic structure,
wherein as the suture attachment device enters the anatomic structure, the prongs radially deflect inward; and
after entering the anatomic structure, the prongs radially deflect outward and secure the suture to the anatomic structure.

14. The method of claim 13, further comprising passing the suture through a plurality of cannulated members of a plurality of suture attachment devices.

15. The method of claim 14, further comprising tightening the suture to nest each of the suture attachment devices together.

16. The method of claim 15, wherein the prongs of each suture attachment device are configured to nest together.

17. The method of claim 13, further comprising passing a plurality of the sutures through a plurality of the cannulated members of a plurality of suture attachment devices.

18. The method of claim 17, further comprising knotting each of the sutures to secure the sutures to a respective suture attachment device; and
passing each of the suture attachment device with the respective suture secured thereto into the anatomic structure,
wherein as the suture attachment devices enter the anatomic structure, the prongs radially deflect inward; and
after entering the anatomic structure, the prongs radially deflect outward to secure the sutures to the anatomic structure.

19. The method of claim 18, further comprising tightening the sutures to nest each of the suture attachment devices together.

20. The method of claim 19, wherein the prongs of each suture attachment device are configured to nest together.

21. A suture attachment device for securing a suture to bone, comprising:
a cannulated member including a first surface and a second surface;
a plurality of deflectable first prongs extending axially and flaring radially outward from the first surface, the plurality of first prongs defining a sharpened end of the suture attachment device; and
a plurality of second prongs extending axially outward from the second surface, the plurality of second prongs defining a blunt end of the suture attachment device;
wherein the plurality of first prongs and the plurality of second prongs define nesting features for engaging a plurality of the suture attachment devices together.

22. The suture attachment device according to claim 21, wherein each prong of the first and second prongs includes a first tapered surface and a second tapered surface that extend from the proximal end to the distal end and terminate at an apex.

23. The suture attachment device of claim 22, wherein the apex of each first prong is sharp, and the apex of each second prong is blunt.

24. The suture attachment device according to claim 22, wherein the first tapered surface and the second tapered surface each include a radius of curvature.

25. The suture attachment device according to claim 21, wherein the first prongs have a greater length than the second prongs.

26. The suture attachment device according to claim 21, wherein an outer diameter defined by the first prongs is greater than an outer diameter defined by the second prongs.
